(19) United States
(12) Patent Application Publication
(10) Pub. No.: US 2005/0197588 A1
(43) Pub. Date: Sep. 8, 2005

(54) Sleep disordered breathing alert system

(76) Inventor: Scott Freeberg, White Bear Lake, MN (US)

Correspondence Address: CRAWFORD MAUNU PLLC, 1270 NORTHLAND DRIVE, SUITE 390, ST. PAUL, MN, 55120, US

(21) Appl. No.: 10/793,177

(22) Filed: Mar. 4, 2004

(51) Int. Cl. A61B 5/08; A61N 1/18; A61N 1/20; A61N 1/22

(52) U.S. Cl. 600/529; 128/903; 607/6

(57) ABSTRACT

A sleep disordered breathing alert system includes an implantable device configured to detect sleep disordered breathing in a patient. If the patient experiences disordered breathing during sleep, the implantable device transmits a signal to a patient-external device. The patient-external device receives the signal and generates an alert responsive to the detection of the sleep disordered breathing. The alert may be a vibration, an audible alert, a visual display, or other indicator.

### FIELD OF THE INVENTION

The present invention relates generally to methods and systems for generating a sleep disordered breathing alert.

### BACKGROUND OF THE INVENTION

Disordered breathing may be caused by a wide spectrum of respiratory conditions involving the disruption of the normal respiratory cycle. Although disordered breathing often occurs during sleep, the condition may also occur while the patient is awake. Respiratory disruption can be particularly serious for patients concurrently suffering from cardiovascular deficiencies, such as congestive heart failure. Unfortunately, disordered breathing is often undiagnosed. If left untreated, the effects of disordered breathing may result in serious health consequences for the patient.

Various types of disordered respiration have been identified, including, for example, apnea, hypopnea, dyspnea, hyperpnea, tachypnea, and periodic breathing, including Cheyne-Stokes respiration (CSR). Apnea is a fairly common disorder characterized by periods of interrupted breathing. Apnea is typically classified based on its etiology. One type of apnea, denoted obstructive apnea, occurs when the patient's airway is obstructed by the collapse of soft tissue in the rear of the throat. Central apnea is caused by a derangement of the central nervous system control of respiration. The patient ceases to breathe when control signals from the brain to the respiratory muscles are absent or interrupted. Mixed apnea is a combination of the central and obstructive apnea types. Regardless of the type of apnea, people experiencing an apnea event stop breathing for a period of time. The cessation of breathing may occur repeatedly during sleep, sometimes hundreds of times a night and sometimes for a minute or longer.

Sleep apnea is particularly dangerous for infants and patients with severe cardiopulmonary deficiencies such as those associated with chronic heart failure. Due to the potential serious consequences of interrupted respiration, methods and systems for detecting and alleviating sleep disordered breathing is of particular interest.

### SUMMARY OF THE INVENTION

The present invention is directed to systems and methods for generating a sleep disordered breathing alert.

In one embodiment of the invention, a sleep disordered breathing alert system includes a sensing system configured to sense one or more conditions associated with sleep disordered breathing. The system further includes an implantable device and a patient-external device. The implantable device incorporates a processor configured to detect disordered breathing occurring during sleep. The implantable device also includes a transmitter configured to transmit a signal if the sleep disordered breathing is detected. The patient-external device is configured to receive the signal and generate an alert responsive to the detection of the sleep disordered breathing.

Another embodiment of the invention involves a method for generating a sleep disordered breathing alert. The method includes detecting that a patient is asleep and detecting disordered breathing while the patient is asleep. One or both of detecting sleep and detecting the disordered breathing are performed implantably. An alert responsive to the detection of the sleep disordered breathing is generated.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a flowchart of a method of generating a sleep disordered breathing alert in accordance with embodiments of the invention;

FIG. 2A is a partial view of an implantable device including a system for detecting sleep and/or sleep disordered breathing in accordance with embodiments of the invention;

FIG. 2B is a diagram illustrating an implantable transthoracic cardiac sensing and/or stimulation (ITCS) device that may be used in connection with a sleep disordered breathing alert system in accordance with embodiments of the invention;

FIG. 3 is a block diagram of a medical system including a medical device configured to detect sleep and/or sleep disordered breathing in accordance with embodiments of the invention;

FIG. 4 is a graph of a normal respiration signal measured by a transthoracic impedance sensor that may be utilized for sleep detection and/or disordered breathing detection in accordance with embodiments of the present invention;

FIG. 5 illustrates a graph of a patient activity signal derived from an accelerometer positioned to detect patient movement over a 24 hour period that may be utilized for sleep detection in accordance with embodiments of the invention;

FIG. 6 illustrates graphs of minute ventilation signals derived from a transthoracic impedance sensor that may be utilized for sleep detection in accordance with embodiments of the invention;

FIG. 7 is a graph depicting modification of a sleep threshold associated with a patient activity based on changes in the patient's minute ventilation in accordance with embodiments of the invention;

FIG. 8 is a graph illustrating respiration intervals used for disordered breathing detection according to embodiments of the invention;

FIG. 9 is a graph illustrating detection of sleep apnea and severe sleep apnea in accordance with embodiments of the invention; and

FIGS. 10A-10F are conceptual diagrams of a sleep disordered breathing alert system in accordance with embodiments of the invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DESCRIPTION OF VARIOUS EMBODIMENTS

In the following description of the illustrated embodiments, references are made to the accompanying drawings that form a part hereof, and in which are shown by way of illustration, various embodiments by which the invention may be practiced. It is to be understood that other embodiments may be utilized, and structural and functional changes may be made without departing from the scope of the present invention.

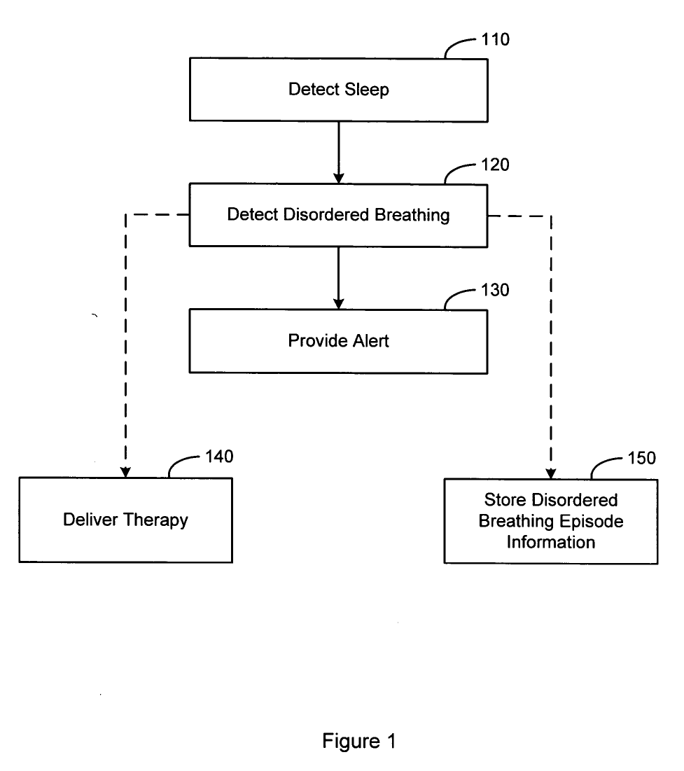

Embodiments of the present invention are directed to systems and methods for monitoring patient breathing and generating an alert if the patient experiences disordered breathing during sleep. FIG. 1 is a flowchart of a method of generating a sleep disordered breathing alert in accordance with embodiments of the invention. The method involves detecting 110 when the patient is asleep and detecting 120 a disordered breathing during sleep.

The patient's sleep state may be determined by analyzing one or more patient conditions indicative of sleep. For example, sleep may be detected on the basis of changes in the patient's heart rate, activity, respiration, or a combination of these conditions and/or other conditions. The conditions used to detect sleep may be sensed using a combination of implantable or patient-external sensors and devices.

After determining the patient is asleep, the system monitors one or more respiration-related signals to detect sleep disordered breathing. Disordered breathing may be detected by sensing and analyzing various physiological and/or non-physiological conditions associated with disordered breathing. Detection of disordered breathing may involve comparing one condition or multiple conditions to one or more thresholds or other indices indicative of disordered breathing.

In one embodiment, disordered breathing is detected by analyzing the patient's respiration patterns as described in more detail below. Patient respiration may be sensed using an implanted or patient-external sensor. For example, implantable methods of sensing patient respiration may involve the use of an implantable transthoracic impedance sensor and/or an implantable blood gas sensor. Patient-external methods of sensing patient respiration may involve the use of devices such as a respiratory belt or external air-flow meter.

If disordered breathing is detected 120 during sleep, an alert is generated 130. The alert may comprise, for example, a visual display, an auditory tone, a vibration, and/or other appropriate indicators. The alert may be generated immediately or otherwise contemporaneously with the sleep disordered breathing.

In one scenario, the alert is directed to the patient, for example, to awaken the patient from sleep and thus end the sleep apnea episode. In another scenario, the alert is directed to the patient's caregiver, so that the caregiver can wake the patient or provide an appropriate therapy, for example. In one implementation, a signal may be transmitted from an implantable device to a patient monitoring station used by the patient's caregiver. The patient monitoring station may generate and alert, e.g., an audible alarm or visual alarm, responsive to the detection of the sleep disordered breathing.

In yet another scenario, the sleep disordered breathing alert system dials a remote device such as a mobile communications device upon detection of sleep disordered breathing. The mobile communications device may comprise a cell phone, pager, personal data assistant (PDA), or other communications device, for example. The mobile communications device may generate an appropriate alert responsive to the detection of sleep disordered breathing.

The system may be configured to provide a number of types of alert. For example, a first tone or vibration intensity may be generated when a sleep apnea episode is detected. A second tone or vibration intensity may be generated when a severe sleep apnea episode is detected.

Detection of the sleep disordered breathing may optionally initiate the delivery 140 of an appropriate therapy to alleviate the disordered breathing. Various types of therapies have been used to treat sleep disordered breathing.

In one implementation, detection of sleep disordered breathing may trigger the application of cardiac electrical stimulation therapy for disordered breathing. Methods and systems for providing cardiac electrical stimulation therapy for sleep disordered breathing are described in commonly owned U.S. patent application Ser. No. 10/643,203 (Docket Number GUID.059PA), filed Aug. 18, 2003 and incorporated herein by reference in its entirety.

In another implementation, detection of sleep disordered breathing may be used to initiate an externally delivered respiration therapy. A commonly prescribed treatment for sleep apnea is continuous positive airway pressure (CPAP). A CPAP device delivers air pressure through a nasal mask worn by the patient. The application of continuous positive airway pressure keeps the patient's throat open, reducing or eliminating the obstruction causing the apnea. In one embodiment of the invention, detection of sleep disordered breathing may initiate or modify CPAP therapy delivered to the patient.

In a further implementation, both cardiac pacing and positive airflow pressure therapy may be delivered to the patient. Methods and systems for providing coordinated therapies involving cardiac electrical stimulation therapy and external respiration therapy for the treatment of disordered breathing are described in commonly owned U.S. Patent Application Ser. No. 60/504,561 (Docket No.: GUID.138P1), filed Sep. 18, 2003, and incorporated herein by reference.

In a yet another implementation, detection of sleep disordered breathing may trigger a muscle stimulation therapy. Prolapse of the tongue muscles has been attributed to diminishing neuromuscular activity of the upper airway. A treatment for obstructive sleep apnea involves compensating for the decreased muscle activity by electrical activation of the tongue muscles. The hypoglossal (HG) nerve innervates the protrusor and retractor tongue muscles. An appropriately applied electrical stimulation to the hypoglossal nerve, for example, may prevent backward movement of the tongue, thus preventing the tongue from obstructing the airway.

Another electrical stimulation method for treating disordered breathing involves phrenic nerve pacing, which is also denoted diaphragmatic pacing. The phrenic nerve is generally known as the motor nerve of the diaphragm. It runs through the thorax, along the heart, and then to the diaphragm. Diaphragmatic pacing involves the use of electrical stimulation of the phrenic nerve to control the patient's diaphragm. The electric stimulus of the phrenic nerve causes the diaphragm to induce a respiratory cycle. Methods and systems of diaphragmatic pacing are described in commonly owned U.S. Pat. No. 6,415,183, which is incorporated herein by reference.

Additionally, or alternatively, information about the patient's sleep and/or the detected sleep disordered breathing may be stored 150, either by an implantable device or by a patient-external device. The stored information may be analyzed for diagnostic purposes or to adjust the patient's therapy, for example. Methods and systems involving monitoring various aspects of sleep and respiration are described in commonly owned U.S. patent application Ser. No. 10/642,998 (Docket Number GUID.058PA), filed Aug. 18, 2003, which is incorporated herein by reference.

In accordance with embodiments of the invention, the sleep disordered breathing alert system implantably detects sleep and/or sleep disordered breathing. In a preferred embodiment, the implantable portion of the sleep disordered breathing alert system maybe incorporated within the housing of a cardiac rhythm management system (CRM). The CRM may comprise one or more of a variety of cardiac rhythm devices, such as an implantable cardiac defibrillator (ICD), pacemaker, and/or cardiac resychronizer, for example.

The implantable sleep detector and/or sleep disordered breathing detector may be coupled to one or more sensors and/or other devices configured to sense patient conditions indicative of sleep and/or sleep disordered breathing. In various embodiments, the sensors and/or other devices may be fully or partially implantable or patient-external (i.e., not invasively implanted within the patient's body). A patient-external medical device or sensor may be positioned on the patient, near the patient, or in any location external to the patient. It is understood that a portion of a patient-external medical device or sensor may be positioned within an orifice of the body, such as the nasal cavity or mouth, yet can be considered external to the patient (e.g., mouth pieces/appliances, tubes/appliances for nostrils, or temperature sensors positioned in the ear canal). The sensors and/or other devices may be coupled to the implantable portion of the sleep disordered breathing alert system using wired or wireless communication links.

The implantable portion of the sleep disordered breathing alert system determines that the patient is asleep and/or detects sleep disordered breathing. Upon detection of sleep disordered breathing, the implantable device may transmit a signal to a patient-external device. The patient-external device then generates an alert. The alert may function to awake the patient, and/or to notify the patient's caregiver that the patient is experiencing sleep disordered breathing.

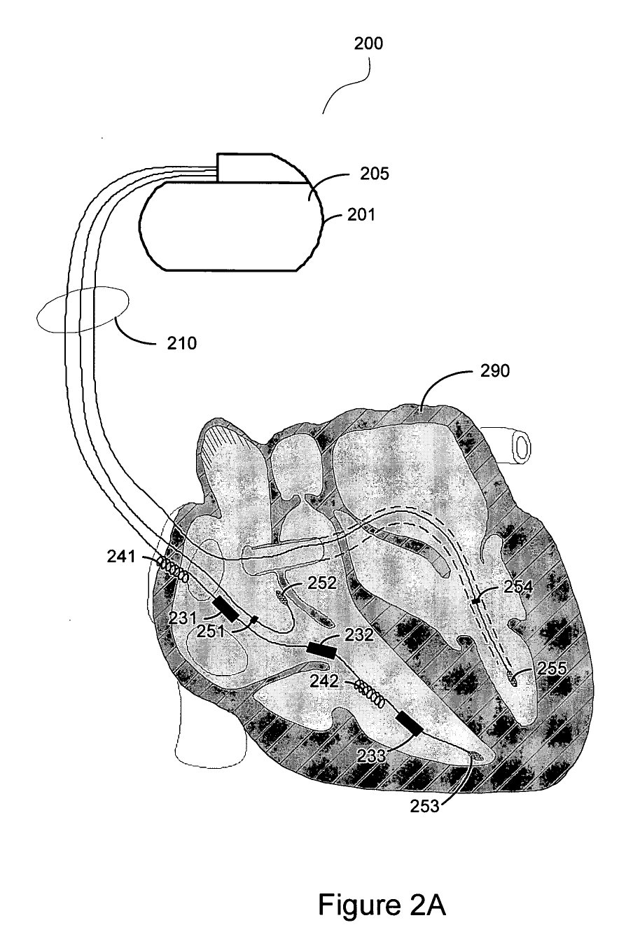

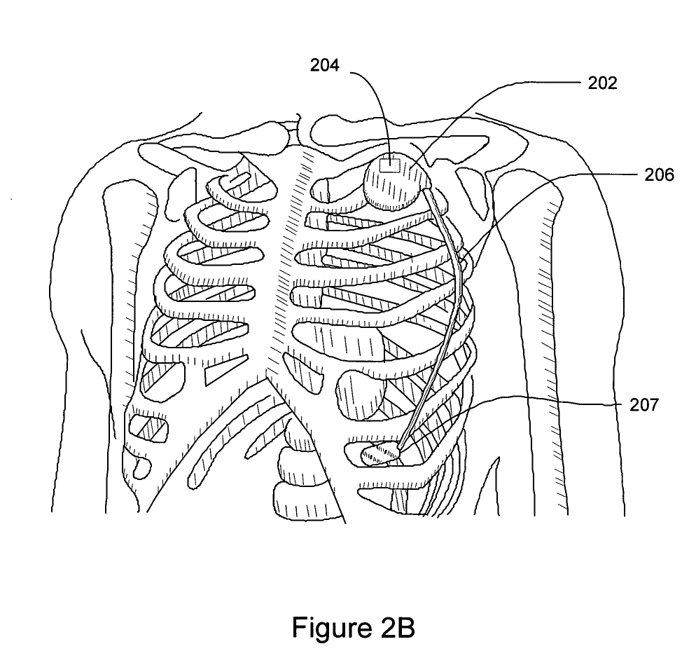

FIG. 2A is a partial view of an implantable device including a system for detecting sleep and/or sleep disordered breathing in accordance with embodiments of the invention. The implantable device illustrated in FIG. 2 comprises a cardiac rhythm management device (CRM) including portions of a sleep disordered breathing alert system. The CRM 200 includes an implantable pulse generator 205 electrically and physically coupled to an intracardiac lead system 210. Portions of the intracardiac lead system 210 are inserted into the patient's heart 290. The intracardiac lead system 210 includes one or more electrodes 231-233, 241-242, 251-255 configured to sense electrical cardiac activity of the heart, deliver electrical stimulation to the heart, and/or to sense the patient's transthoracic impedance. Portions of the housing 201 of the pulse generator 205 may optionally serve as a can electrode.

Communications circuitry is disposed within the housing 201 for facilitating communication between the pulse generator 205 and an external device, such as a portable or bed-side station, patient-carried/worn device, or an external programmer, for example. The external device may provide the alert signal, or serve as a repeater, transmitting the sleep disordered breathing information to a separate device. The communications circuitry can also facilitate unidirectional or bidirectional communication with one or more external, cutaneous, or subcutaneous physiologic or non-physiologic sensors, patient-input devices and/or information systems.

The lead system 210 of the CRM 200 may incorporate one or more transthoracic impedance electrodes 231-233 that may be used to sense the patient's respiration. The impedance electrodes 231-233 may be coupled to impedance drive/sense circuitry 230 positioned within the housing 201 of the pulse generator 205.

In one implementation, impedance drive/sense circuitry within the pulse generator 205 generates a current that flows through the tissue between an impedance drive electrode 233 and a can electrode on the housing 201 of the pulse generator 205. The voltage at an impedance sense electrode 231, 232 relative to the can electrode changes as the patient's transthoracic impedance changes. The voltage signal developed between the impedance sense electrode 231, 232 and the can electrode is detected by the impedance sense circuitry.

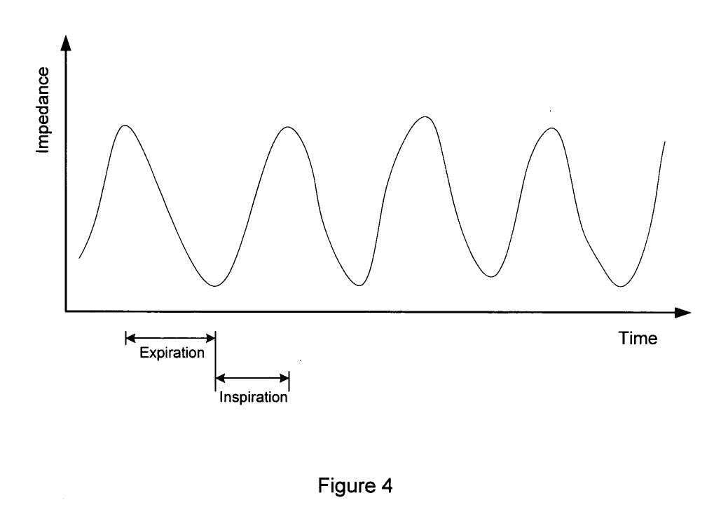

The voltage signal developed at the impedance sense electrode, 231, 232 illustrated in FIG. 4, is proportional to the patient's transthoracic impedance. Transthoracic impedance increases during respiratory inspiration and decreases during respiratory expiration. The area of the impedance signal over one breath cycle is proportional to the volume of air moved in one breath, denoted the tidal volume. The volume of air moved per minute is denoted the minute ventilation.

The lead system 210 may include one or more pace/sense electrodes 251-255 positioned in one or more heart chambers for sensing electrical signals from the patient's heart 290 and/or delivering pacing pulses to the heart 290. The sense/pace electrodes 251-255 may be used to sense and pace one or more chambers of the heart, including the left ventricle, the right ventricle, the left atrium and/or the right atrium. The lead system 210 may include one or more defibrillation electrodes 241, 242 for delivering defibrillation/cardioversion shocks to the heart.

The pulse generator 205 may include circuitry for detecting cardiac arrhythmias and for providing therapy in the form of electrical stimulation delivered to the heart through the lead system 210. The pulse generator 205 may also incorporate a sleep detector and/or a disordered breathing detector, as described in more detail below. Although methods for sensing respiration described herein involve transthoracic impedance measurements, other methods of acquiring respiration signals are also possible. For example, a respiration signal may be acquired using airflow measurements, signals from a respiratory belt, blood gas measurements, and/or other methods.

FIG. 2B is a diagram illustrating another configuration of an implantable medical device that may be used in connection with a sleep disordered breathing alert system in accordance with embodiments of the invention. The implantable device illustrated in FIG. 2B is an implantable transthoracic cardiac sensing and/or stimulation (ITCS) device that may be implanted under the skin in the chest region of a patient. The ITCS device may, for example, be implanted subcutaneously such that all or selected elements of the device are positioned on the patient's front, back, side, or other body locations suitable for sensing cardiac activity and delivering cardiac stimulation therapy. It is understood that elements of the ITCS device may be located at several different body locations, such as in the chest, abdominal, or subclavian region with electrode elements respectively positioned at different regions near, around, in, or on the heart.

The ITCS device may incorporate circuitry to detect sleep disordered breathing. Portions of the sleep disordered breathing circuitry 204 may be positioned within the primary housing of the ITCS device. The primary housing (e.g., the active or non-active can) of the ITCS device, for example, may be configured for positioning outside of the rib cage at an intercostal or subcostal location, within the abdomen, or in the upper chest region (e.g., subclavian location, such as above the third rib). In one implementation, one or more electrodes may be located on the primary housing and/or at other locations about, but not in direct contact with the heart, great vessel or coronary vasculature.

In another implementation, one or more electrodes may be located in direct contact with the heart, great vessel or coronary vasculature, such as via one or more leads implanted by use of conventional transvenous delivery approaches. In another implementation, for example, one or more subcutaneous electrode subsystems or electrode arrays may be used to sense cardiac activity and deliver cardiac stimulation energy in an ITCS device configuration employing an active can or a configuration employing a non-active can. Electrodes may be situated at anterior and/or posterior locations relative to the heart.

In particular configurations, the ITCS device may perform functions traditionally performed by cardiac rhythm management devices, such as providing various cardiac monitoring, pacing and/or cardioversion/defibrillation functions. Exemplary pacemaker circuitry, structures and functionality, aspects of which can be incorporated in an ITCS device of a type that may benefit from multi-parameter sensing configurations, are disclosed in commonly owned U.S. Pat. Nos. 4,562,841; 5,284,136; 5,376,476; 5,036,849; 5,540,727; 5,836,987; 6,044,298; and 6,055,454, which are hereby incorporated herein by reference in their respective entireties. It is understood that ITCS device configurations can provide for non-physiologic pacing support in addition to, or to the exclusion of, bradycardia and/or anti-tachycardia pacing therapies. Exemplary cardiac monitoring circuitry, structures and functionality, aspects of which can be incorporated in an ITCS of the present invention, are disclosed in commonly owned U.S. Pat. Nos. 5,313,953; 5,388,578; and 5,411,031, which are hereby incorporated herein by reference in their respective entireties.

In FIG. 2B, there is shown a configuration of a transthoracic cardiac sensing and/or stimulation (ITCS) device having components implanted in the chest region of a patient at different locations. In the particular configuration shown in FIG. 2B, the ITCS device includes a primary housing 202 within which various sensing, detection, processing, and energy delivery circuitry can be housed. An ITCS device can incorporate circuitry, structures and functionality of the subcutaneous implantable medical devices disclosed in commonly owned U.S. Pat. Nos. 5,203,348; 5,230,337; 5,360,442; 5,366,496; 5,397,342; 5,391,200; 5,545,202; 5,603,732; and 5,916,243 and commonly owned U.S. patent applications “Subcutaneous Cardiac Sensing, Stimulation, Lead Delivery, and Electrode Fixation Systems and Methods,” Ser. No. 60/462,272, filed Apr. 11, 2003, and Hybrid Transthoracic/lntrathoracic Cardiac Stimulation Devices and Methods,” Ser. No. 10/462,001, filed Jun. 13, 2003, and “Methods and Systems Involving Subcutaneous Electrode Positioning Relative to A Heart,” Ser. No. 10/465,520, filed Jun. 19, 2003 which are incorporated by reference.

Portions of the circuitry used to detect sleep disordered breathing may be positioned within or on the primary housing 202, on the lead assembly 206, or on the subcutaneous electrode assembly 207. For example, a sleep detector and a disordered breathing detector may be located within the primary housing 202. Electrodes for sensing transthoracic impedance positioned on the primary housing 202, on the lead assembly 206, and/or on the subcutaneous electrode assembly 207.

In one configuration, the ITCS includes an impedance sensor configured to generate a signal corresponding to patient respiration used to detect sleep disordered breathing. The impedance sensor may include impedance drive/sense circuitry coupled to impedance electrodes. The impedance drive circuitry generates a current that flows between a subcutaneous impedance drive electrode and a can electrode on the primary housing 202 of the ITCS device. The voltage at a subcutaneous impedance sense electrode relative to the can electrode changes as the patient's transthoracic impedance changes. The voltage signal developed between the impedance sense electrode and the can electrode is detected by the impedance sense circuitry.

As previously discussed, the transthoracic impedance signal is related to patient respiration, with impedance increasing during respiratory inspiration and decreasing with respiratory expiration. Respiration signals generated by the transthoracic impedance sensor may be used to detect disordered breathing.

Communications circuitry is disposed within the housing 202 for facilitating communication between the ITCS device and an external communication device, such as a portable or bed-side communication station, patient-carried/worn communication station, or external programmer, for example. The communications circuitry can also facilitate unidirectional or bidirectional communication with one or more external, cutaneous, or subcutaneous physiologic or non-physiologic sensors. The housing 202 is typically configured to include one or more electrodes (e.g., can electrode and/or indifferent electrode).

In the configuration shown in FIG. 2, a subcutaneous electrode assembly 207 can be positioned under the skin in the chest region and situated distal from the housing 202. The subcutaneous and, if applicable, housing electrode(s) can be positioned about the heart at various locations and orientations, such as at various anterior and/or posterior locations relative to the heart. The subcutaneous electrode assembly 207 is coupled to circuitry within the housing 202 via a lead assembly 306. One or more conductors (e.g., coils or cables) are provided within the lead assembly 206 and electrically couple the subcutaneous electrode assembly 207 with circuitry in the housing 202. One or more transthoracic impedance electrodes along with one or more cardiac sense, sense/pace or defibrillation electrodes can be situated on the elongated structure of the lead assembly 206, the housing 202, and/or the distal electrode assembly (shown as subcutaneous electrode assembly 207 in the configuration shown in FIG. 2B).

Various methods and systems related to implantable transthoracic cardiac sensing and stimulation devices are described in commonly owned U.S. patent applications “Subcutaneous Cardiac Sensing, Stimulation, Lead Delivery, and Electrode Fixation Systems and Methods,” Ser. No. 60/462,272, filed Apr. 11, 2003, and Hybrid Transthoracic/lntrathoracic Cardiac Stimulation Devices and Methods,” Ser. No. 10/462,001, filed Jun. 13, 2003, and “Methods and Systems Involving Subcutaneous Electrode Positioning Relative to A Heart,” Ser. No. 10/465,520, filed Jun. 19, 2003 and U.S. Pat. Nos. 5,203,348, 5,230,337, 5,360,442, 5,366,496, 5,397,342, 5,391,200, 5,545,202, 5,603,732, 5,916,243 previously incorporated herein by reference.

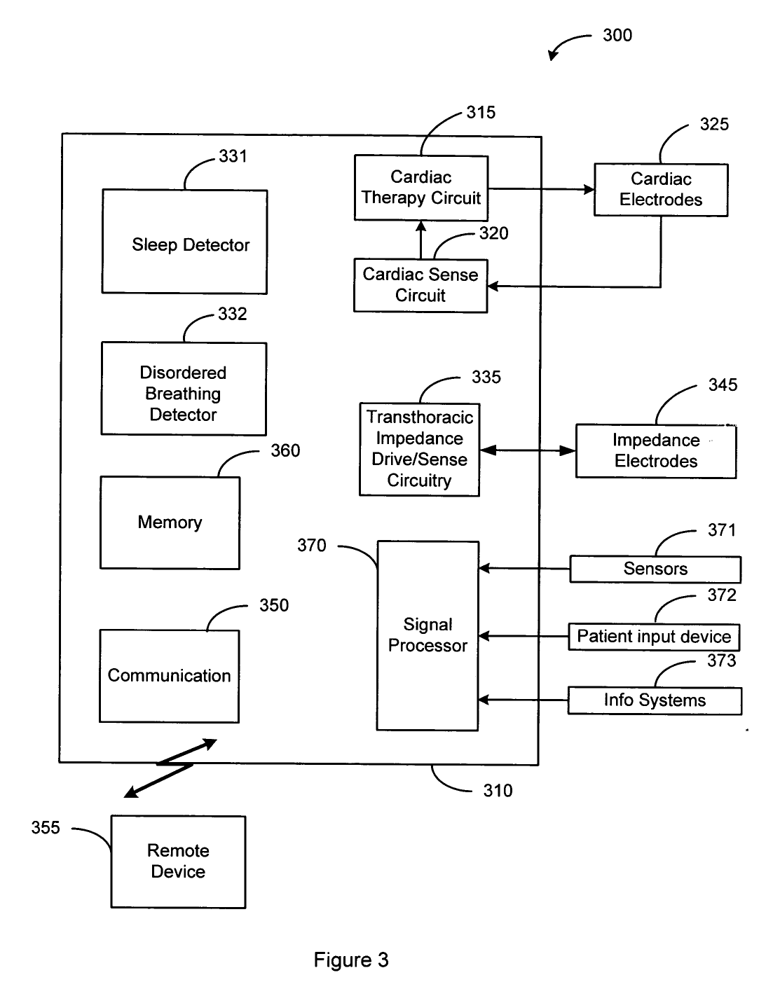

FIG. 3 is a block diagram of a medical system 300 including a medical device 310 configured to detect sleep and/or sleep disordered breathing in accordance with embodiments of the invention. The medical device 310 may be coupled to a sensing system comprising various sensors, 325, 345, 371 patient input devices 372, and information systems 373. The sensors, 325, 345, 371, patient input device 372, and information system 373 may acquire information used in the determination sleep and/or sleep disordered breathing. The medical device 310 and the sensors 325, 345, 371 may be fully or partially implantable.

In a preferred embodiment, the medical device 310 includes a cardiac therapy circuit 315 and a cardiac sense circuit 320 coupled through a lead system to cardiac electrodes 325 positioned in, on or about the patient's heart. The cardiac electrodes 325 are electrically coupled to the patient's heart for sensing electrical cardiac signals and delivering therapy to the heart in the form of electrical stimulation energy, e.g., pacing pulses and/or defibrillation/cardioversion shocks.

The medical device 310 incorporates a sleep detector 331 and a disordered breathing detector 332. In one embodiment, disordered breathing is detected through analysis of the patient's respiration signal. A respiration signal may be acquired, for example, based on the patient's transthoracic impedance measurements as described in connection with FIG. 2A or 2B. Other methods of acquiring a respiratory waveform, such as, air-flow measurements, blood gas measurements, and/or patient chest wall motions are also possible.

In the embodiment illustrated in FIG. 3, the medical device 310 incorporates transthoracic impedance sensing circuitry comprising drive/sense circuitry 335 coupled to a number of impedance electrodes 345 that may be positioned intrathoracicly, subcutaneously, or externally with respect to the patient's chest. The transthoracic impedance sensing circuitry detects modulation of the patient's transthoracic impedance as the patient inhales and exhales. Signals from the impedance drive/sense circuitry 335 may be used by a sleep detector 332 to determine when the patient is asleep. Additionally or alternatively, the signals from the impedance drive/sense circuitry 335 may be used by a disordered breathing detector 331 to detect disordered breathing.

As previously mentioned, one or more patient conditions indicative of sleep and/or disordered breathing may be acquired using the cardiac electrodes 325, sensors 371, patient input devices 372 and/or other information systems 373. The one or more patient conditions may be used in connection with sleep detection and/or disordered breathing detection in addition to, or as an alternative to, the respiration signal acquired in accordance with a transthoracic impedance sensing methodology discussed above. Patient conditions related to sleep and/or sleep disordered breathing may include both physiological and non-physiological contextual conditions affecting the patient. Physiological conditions may include a broad category of conditions associated with the internal functioning of the patient's physiological systems, including the cardiovascular, respiratory, nervous, muscle and other systems. Examples of physiological conditions include blood chemistry, patient activity, respiration quality, sleep quality, among others.

Contextual conditions generally encompass non-physiological, patient-external or background conditions. Contextual conditions may be broadly defined to include, for example, present environmental conditions, such as ambient temperature, humidity, and air pollution index. Contextual conditions may also include historical/background conditions relating to the patient, including the patient's normal sleep time and the patient's medical history, for example. Methods and systems for detecting some contextual conditions, including, for example, proximity to bed detection, are described in commonly owned U.S. patent application entitled “Methods and Devices for Detection of Context When Addressing A Medical Condition of a Patient,” Ser. No. 10/269611, filed Oct. 11, 2002, which is incorporated by reference herein in its entirety.

A representative set of conditions that may be used for detecting sleep and/or disordered breathing is provided in Table 1. Table 1 also provides illustrative sensing methods that may be employed to sense the conditions. The list of conditions and sensing methods in Table 1 is not exhaustive and other conditions may additionally be utilized.

TABLE 1

| | | | Sensor type or Detection |
|---|---|---|---|
| Condition Type | | Condition | method |
| Physiological | Cardiovascular | Heart rate | EGM, EGG |
| | System | Heart rate variability | |
| | | QT interval | |
| | | Ventricular filling | Intracardiac pressure |
| | | pressure | sensor |
| | | Blood pressure | Blood pressure sensor |
| | Respiratory | Snoring | Accelerometer |
| | System | | Microphone |
| | | Respiration pattern | Transthoracic |
| | | (Tidal volume Minute | impedance sensor (AC) |
| | | ventilation Respiratory | |
| | | rate) | |
| | | Patency of upper airway | Intrathoracic impedance |
| | | | sensor |
| | | Pulmonary congestion | Transthoracic |
| | | | impedance sensor (DC) |
| | Nervous System | Sympathetic nerve | Muscle sympathetic |
| | | activity | nerve Activity sensor |
| | | Brain activity | EEG |
| | Blood Chemistry | CO2 saturation | Blood analysis |
| | | O2 saturation | |
| | | Blood alcohol content | |
| | | Adrenalin | |
| | | Brain Natriuretic Peptide | |
| | | (BMP) | |
| | | C-Reactive Protein | |
| | | Drug/Medication/Tobacco | |
| | | use | |
| | Muscle System | Muscle atonia | EMG |
| | | Eye movement | EOG |
| | | Patient activity | Accelerometer, MV, etc. |
| | | Limb movements | Accelerometer, EMG |
| | | Jaw movements | Accelerometer, EMG |
| | | Posture | Multi-axis accelerometer |
| Contextual | Environmental | Ambient temperature | Thermometer |
| | | Humidity | Hygrometer |
| | | Pollution | Air quality website |
| | | Time | Clock |
| | | Barometric pressure | Barometer |
| | | Ambient noise | Microphone |
| | | Ambient light | Photodetector |
| | | Altitude | Altimeter |
| | | Location | GPS, proximity sensor |
| | | Proximity to bed | Proximity to bed sensor |
| | Historical/ | Historical sleep time | Patient input, previously |
| | Background | | detected sleep onset |
| | | | times |
| | | Medical history | Patient input |
| | | Age | |
| | | Recent exercise | |
| | | Weight | |
| | | Gender | |
| | | Body mass index | |
| | | Neck size | |
| | | Emotional state | |
| | | Psychological history | |
| | | Daytime sleepiness | |
| | | Patient perception of | |
| | | sleep quality | |
| | | Drug, alcohol, nicotine | |
| | | use | |

Signal processing circuitry 370 within the medical device 310 may be used to condition the signals received from the sensors 371, patient input devices 372, and/or information systems 373. The signal processing circuitry 370 may include, for example, amplifiers, drivers, filters, samplers, A/D converters, and/or other circuitry for processing the signals produced by the sensors 371, patient input devices 372, and/or information systems 373. The medical device 310 may be coupled to the sensors 371, patient input devices 372, and/or information systems 373 through wired or wireless communications links.

The sensors 371 may comprise patient-internal and/or patient-external sensors coupled through leads or wirelessly to the medical device 310 that sense various physiological or non-physiological conditions. The patient input device 372 allows the patient to input information relevant to sleep and/or disordered breathing detection. For example, the patient input device 372 may be particularly useful for inputting information concerning patient activities or perceptions, such as tobacco use, drug use, recent exercise, perceptions of sleep quality, and/or other conditions that are not automatically sensed or detected by the medical system 300.

The medical device 310 may also be coupled to other information systems 373, such as network-connected servers. The medical device 310, may access the information systems 373 to acquire information related to disordered breathing, such as information about conditions associated with an increased or decreased risk of disordered breathing in the patient. For example, the medical device 310 may access an air quality website to acquire the ambient pollution index used in disordered breathing detection.

The medical device 310 may incorporate communication circuitry 350 for transmitting an alert signal to a remote device 355 upon the detection of disordered breathing. The remote device 355 may comprise a portable or bed-side station, patient-carried/worn device, or an external programmer, for example. The communication circuitry 350 may also facilitate transmission of stored information and/or therapy parameters between the medical device 310 and the remote device 355.

The medical device 310 may include a memory circuit 360 that may be used to store information related to disordered breathing. Stored information may include, for example, the date/time, severity, duration, and/or frequency of the disordered breathing. The stored information may be transmitted to a remote device 355, such as a remote device programmer, a patient management server, or other device through a wireless communications link.

Embodiments of the invention involve determining that the patient is asleep prior to detecting disordered breathing. Determining that the patient is asleep may involve comparing changes in one or more patient conditions associated with sleep to thresholds indicative of sleep.

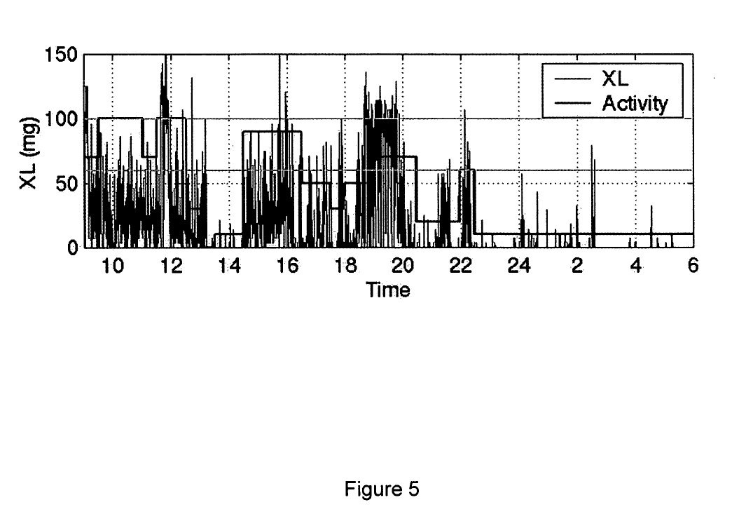

In one example, the patient's activity may be monitored to determine if the patient is asleep. FIG. 5 illustrates a patient activity signal derived from an accelerometer positioned to detect patient movement over a 24 hour period. The accelerometer may be positioned within or on the housing of the CRM device or the ITCS device described in connection with FIGS. 2A and 2B, respectively. The patient activity signal may be filtered or otherwise conditioned to produce an average of patient activity over a time period. FIG. 5 provides representative graphs of the filtered and unfiltered signals. If the filtered activity signal falls below a sleep threshold, then the patient is determined to be asleep.

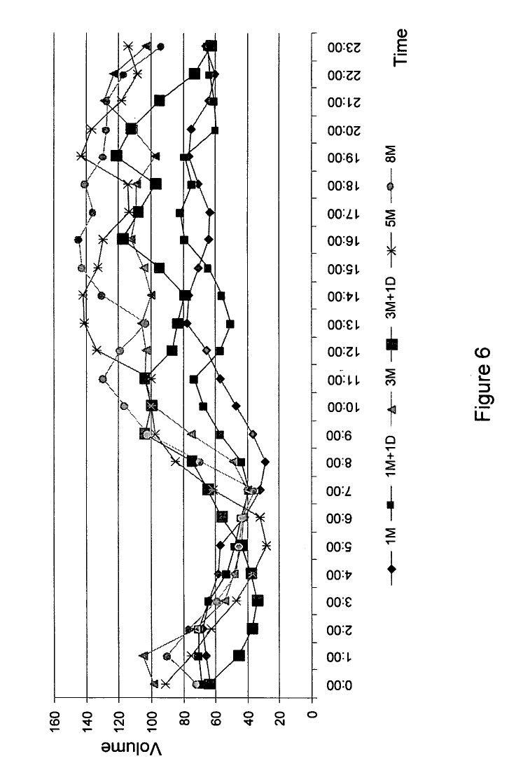

In another example, the patient's minute ventilation signal may be used to determine that the patient is sleeping. FIG. 6 is a composite graph illustrating several graphs of minute ventilation data acquired over a 24 hour period. The patient's minute ventilation signal decreases below a threshold level when the patient is sleeping.

In another example, sleep may be detected by comparing multiple sleep-related conditions to multiple thresholds. For example, the patient may be determined to be asleep if the patient's activity, sensed by an accelerometer, falls below an activity sleep threshold and the patient's heart rate, sensed by cardiac electrodes, falls below a heart rate sleep threshold.

In yet a further example, a sleep threshold used for a first condition may be adjusted by a second condition. The first condition is compared to the adjusted threshold and sleep may be detected based on the comparison. For example, a respiration signal, e.g., minute ventilation signal, may be used to adjust the sleep threshold associated with patient activity. The graph illustrated in FIG. 7 depicts modification of a sleep threshold associated with a patient activity based on changes in the patient's minute ventilation.

An initial sleep threshold 710 may be determined from clinical data of patient activity during sleep acquired from a group of subjects, for example. The initial sleep threshold 710 may alternatively be determined using historical data taken from the particular patient for whom the onset and offset of sleep is to be determined. For example, a history of a particular patient's sleep times can be stored, and a sleep threshold can be developed using data associated with the patient's sleep time history.

Patient activity and minute ventilation are sensed. The initial sleep threshold 710 established for patient activity is adjusted using the minute ventilation signal. For example, if the minute ventilation signal indicates a respiration volume that is incompatible with a sleep state, the sleep threshold of the patient activity may be adjusted downward 730 to require sensing a decreased level of the patient activity before a sleep condition is detected. If the minute ventilation signal indicates a low respiration volume that is compatible with sleep, then the sleep threshold may be adjusted upward 720. Methods and systems for determining whether a patient is asleep are further described in commonly owned U.S. patent application Ser. No. 10/309,771 (Docket Number GUID.064PA), filed Dec. 4, 2002 and incorporated herein by reference.

In accordance with one embodiment, after determining the patient is asleep, the system monitors one or more respiration-related signals to detect sleep disordered breathing. Disordered breathing may be detected by sensing and analyzing various conditions associated with disordered breathing. Table 2 provides examples of how a representative subset of the physiological and/or contextual conditions listed in Table 1 may be used in connection with disordered breathing detection.

Detection of disordered breathing may involve comparing one condition or multiple conditions to one or more thresholds or other indices indicative of disordered breathing. A threshold or other index indicative of disordered breathing may comprise a predetermined level of a particular condition, e.g., blood oxygen level less than a predetermined amount. A threshold or other index indicative of disordered breathing may comprises a change in a level of a particular condition, e.g., heart rate decreasing from a sleep rate to lower rate within a predetermined time interval.

In one approach, the relationships between the conditions may be indicative of disordered breathing. In this embodiment, disordered breathing detection may be based on the existence and relative values associated with two or more conditions. For example, if condition A is present at a level of x, then condition B must also be present at a level of f(x) before a disordered breathing detection is made.

The condition thresholds and/or relationships indicative of disordered breathing may be highly patient specific. The thresholds and/or relationships indicative of disordered breathing may be determined on a case-by-case basis by monitoring conditions affecting the patient and monitoring disordered breathing episodes. The analysis may involve determining levels of the monitored conditions and/or relationships between the monitored conditions associated, e.g., statistically correlated, with disordered breathing episodes. The thresholds and/or relationships used in disordered breathing detection may be updated periodically to track changes in the patient's response to disordered breathing.

TABLE 2

| Condi- | | |
|---|---|---|
| tion | | Examples of how condition may be used in |
| Type | Condition | disordered breathing detection |
| Physio- | Heart rate | Decrease in heart rate may indicate disordered |
| logical | | breathing episode. |
| | | Increase in heart rate may indicate autonomic |
| | | arousal from a disordered breathing episode. |
| | | Decrease in heart rate may indicate the patient |
| | | is asleep. |
| | Heart rate | Disordered breathing causes heart rate |
| | variability | variability to decrease. Changes in HRV |
| | | associated with sleep disordered breathing |
| | | may be observed while the patient is awake or |
| | | asleep |
| | Ventricular | May be used to identify/predict pulmonary |
| | filling | congestion associated with respiratory |
| | pressure | disturbance. |
| | Blood | Swings in on-line blood pressure measures are |
| | pressure | associated with apnea. Disordered breathing |
| | | generally increases blood pressure variability - |
| | | these changes may be observed while the |
| | | patient is awake or asleep. |
| | Snoring | Snoring is associated with a higher incidence |
| | | of obstructive sleep apnea and may be used to |
| | | detect disordered breathing. |
| | Respiration | Respiration patterns including, e.g., respir- |
| | pattern/ | ation rate, may be used to detect disordered |
| | rate | breathing episodes. |
| | | Respiration patterns may be used to determine |
| | | the type of disordered breathing. |
| | | Respiration patterns may be used to detect |
| | | that the patient is asleep. |
| | Patency of | Patency of upper airway is related to |
| | upper | obstructive sleep apnea and may be used to |
| | airway | detect episodes of obstructive sleep apnea. |
| | Pulmonary | Pulmonary congestion is associated with |
| | congestion | respiratory disturbances. |
| | Sympathetic | End of apnea associated with a spike in SNA. |
| | nerve | Changes in SNA observed while the patient is |
| | activity | awake or asleep may be associated with sleep |
| | | disordered breathing |
| | CO2 | Low CO2 levels initiate central apnea. |
| | O2 | O2 desaturation occurs during severe |
| | | apnea/hypopnea episodes. |
| | Blood | Alcohol tends to increase incidence of snoring |
| | alcohol | & obstructive apnea. |
| | content | |
| | Adrenalin | End of apnea associated with a spike in blood |
| | | adrenaline. |
| | BNP | A marker of heart failure status, which is |
| | | associated with Cheyne-Stokes Respiration |
| | C-Reactive | A measure of inflammation that may be related |
| | Protein | to apnea. |
| | Drug/ | These substances may affect the incidence of |
| | Medication/ | both central & obstructive apnea. |
| | Tobacco use | |
| | Muscle | Muscle atonia may be used to detect REM and |
| | atonia | non-REM sleep. |
| | Eye move- | Eye movement may be used to detect REM |
| | ment | and non-REM sleep. |
| Contex- | Temperature | Ambient temperature may be a condition |
| tual | | predisposing the patient to episodes of |
| | | disordered breathing and may be useful in |
| | | disordered breathing detection. |
| | Humidity | Humidity may be a condition predisposing the |
| | | patient to episodes of disordered breathing |
| | | and may be useful in disordered breathing |
| | | detection. |
| | Pollution | Pollution may be a condition predisposing the |
| | | patient to episodes of disordered breathing |
| | | and may be useful in disordered breathing |
| | | detection. |
| | Posture | Posture may be used to confirm or determine |
| | | the patient is asleep. |
| | Activity | Patient activity may be used in relation to |
| | | sleep detection. |
| | Location | Patient location may used to determine if the |
| | | patient is in bed as a part of sleep detection. |
| | Altitude | Lower oxygen concentrations at higher |
| | | altitudes tends to cause more central apnea |

In various implementations, disordered breathing may be detected by analyzing the patient's respiration patterns. Methods and systems of disordered breathing detection based on analysis of respiration patterns are further described in commonly owned U.S. patent application Ser. No. 10/309,770 (Docket Number GUID.054PA), filed Dec. 4, 2002 and incorporated herein by reference.

FIG. 4 illustrates normal respiration as represented by a signal produced by a transthoracic impedance sensor. The transthoracic impedance increases during respiratory inspiration and decreases during respiratory expiration. During non-rapid eye movement sleep, a normal respiration pattern includes regular, rhythmic inspiration—expiration cycles without substantial interruptions.

In one embodiment, disordered breathing may be detected by monitoring the respiratory waveform output of the transthoracic impedance sensor. When the tidal volume (TV) of the patient's respiration, as indicated by the transthoracic impedance signal, falls below a hypopnea threshold, then a hypopnea event is declared. For example, a hypopnea event may be declared if the patient's tidal volume falls below about 50% of a recent average tidal volume or other baseline tidal volume value. If the patient's tidal volume falls further to an apnea threshold, e.g., about 10% of the recent average tidal volume or other baseline value, an apnea event is declared.

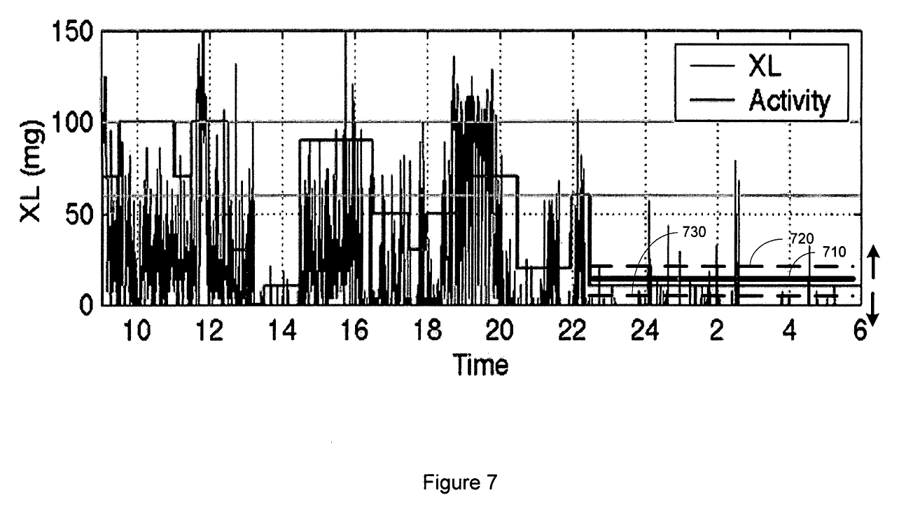

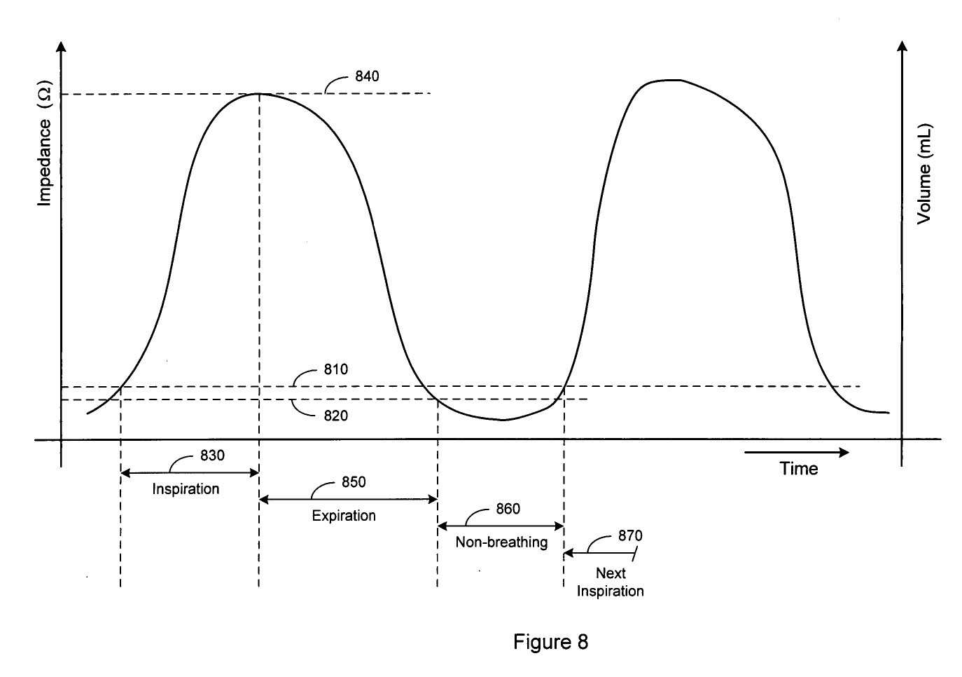

In another embodiment, detection of disordered breathing involves defining and examining a number of respiratory cycle intervals. FIG. 8 illustrates respiration intervals used for disordered breathing detection according to embodiments of the invention. A respiration cycle is divided into an inspiration period corresponding to the patient inhaling, an expiration period, corresponding to the patient exhaling, and a non-breathing period occurring between inhaling and exhaling. Respiration intervals are established using inspiration 810 and expiration 820 thresholds. The inspiration threshold 810 marks the beginning of an inspiration period 830 and is determined by the transthoracic impedance signal rising above the inspiration threshold 810. The inspiration period 830 ends when the transthoracic impedance signal is maximum 840. A maximum transthoracic impedance signal 840 corresponds to both the end of the inspiration interval 830 and the beginning of the expiration interval 850. The expiration interval 850 continues until the transthoracic impedance falls below an expiration threshold 820. A non-breathing interval 860 starts from the end of the expiration period 850 and continues until the beginning of the next inspiration period 870.

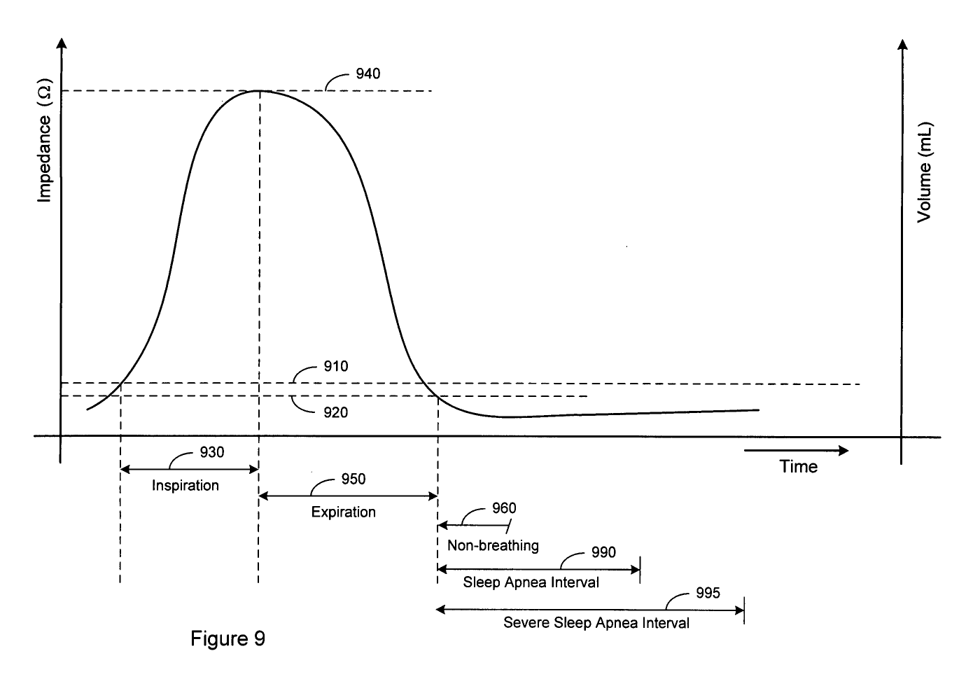

Detection of sleep apnea and severe sleep apnea according to embodiments of the invention is illustrated in FIG. 9. The patient's respiration signals are monitored and the respiration cycles are defined according to inspiration 930, expiration 950, and non-breathing 960 intervals as described in connection with FIG. 8. A condition of sleep apnea is detected when a non-breathing period 960 exceeds a first predetermined interval 990, denoted the sleep apnea interval. A condition of severe sleep apnea is detected when the non-breathing period 960 exceeds a second predetermined interval 995, denoted the severe sleep apnea interval. For example, sleep apnea may be detected when the non-breathing interval exceeds about 10 seconds, and severe sleep apnea may be detected when the non-breathing interval exceeds about 20 seconds.

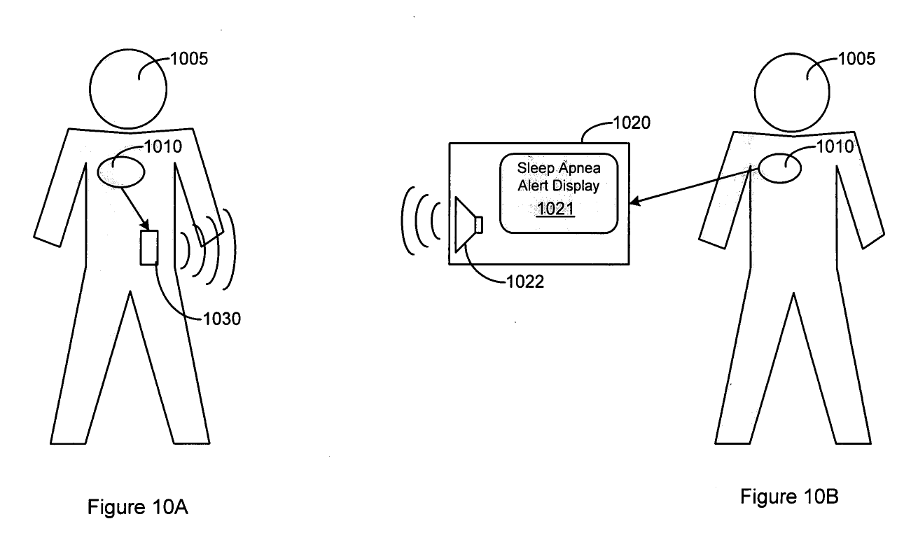

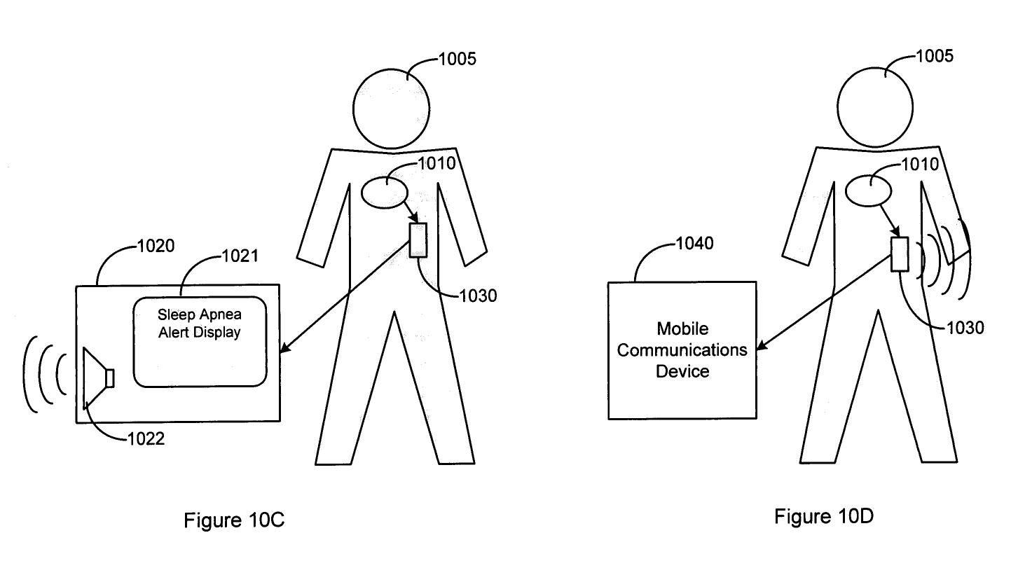

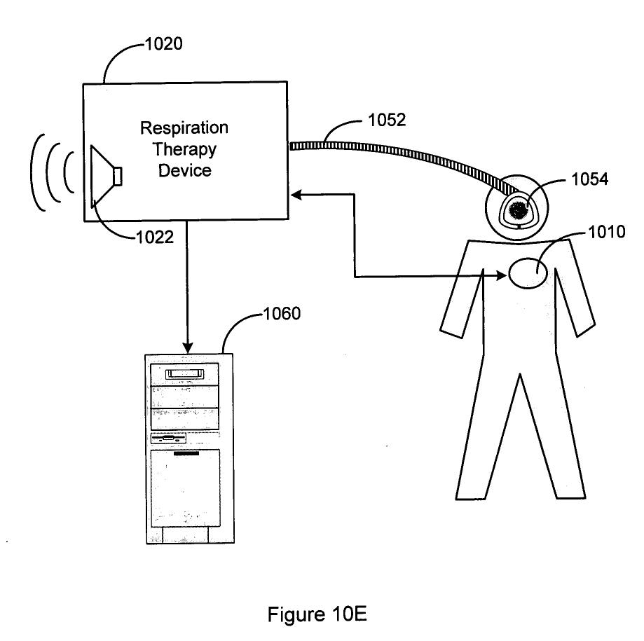

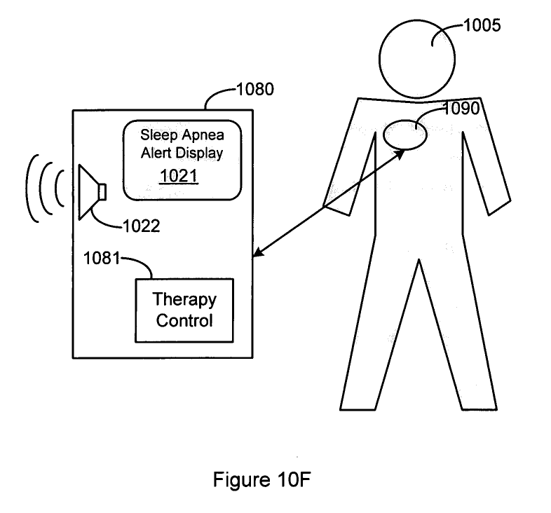

FIGS. 10A-10E conceptually illustrate various configurations of a sleep disordered breathing alert system in accordance with embodiments of the invention. In the embodiments illustrated in FIGS. 10A-10E, an implantable device detects sleep disordered breathing of a patient 1005. As illustrated in FIG. 10A, following detection of sleep disordered breathing, the implantable device 1010 may transmit a signal to a patient-carried or patient-worn device 1030. For example, the patient-worn device may be a relatively small, adhesive-backed device, applied to the patient's chest over or near the location of the implantable device 1010. The patient-worn device 1030 may be equipped with a speaker for producing an alert sound following detection of disordered breathing. The patient-worn device 1030 may alternatively or additionally generate a vibration. In one scenario, the intensity of the vibration and/or the volume of the alert sound may be selected to awaken the patient, thus ending the disordered breathing episode. In another scenario, the alert sound may be chosen to have a volume sufficient to provide an alert to a nearby caretaker that the patient is experiencing disordered breathing.

In one embodiment, the implantable device 1010 is a cardiac rhythm management system. Upon detection of sleep disordered breathing, the CRM system may initiate the delivery of cardiac pacing therapy to mitigate or terminate the sleep disordered breathing. The CRM may store information about the sleep disordered breathing, including, for example, the severity, duration, frequency, and/or date/time of occurrence of the sleep disordered breathing episodes. The stored sleep disordered breathing information may be later transmitted from the implantable device 1010 to a separate device for further analysis.

FIG. 10B illustrates another implementation of a disordered breathing alert system in accordance with embodiments of the invention. In this example, the implantable device 1010 directly transmits a signal to a bed-side monitor 1020 after detecting disordered breathing in the patient 1005. The bed-side monitor 1020 may be equipped with a speaker 1022 for generating an audible alert. Additionally, or alternatively, the bed-side monitor 1020 may comprise a display device 1021 for displaying a visual alert and/or other information. The implantable device 1010 may transmit various information about the sleep disordered breathing episodes, such as date/time of occurrence, severity, or duration to the bedside monitor or to another remote device. The display device 1021 may display the additional information.

FIG. 10C illustrates yet another example of a disordered breathing alert system in accordance with embodiments of the invention. In this example, the implantable device 1010 transmits a signal to patient-worn device 1030 after detecting disordered breathing in the patient 1005. The patient-worn device 1030 operates as a repeater, transmitting the disordered breathing information to a bed-side monitor 1020. The bed-side monitor 1020 and/or the patient-worn device 1030 may be equipped with a speaker 1022 for generating an audible alert. The bed-side monitor 1020 may comprise a display device 1021 for displaying a visual alert and/or other information about the disordered breathing.

FIG. 10D illustrates a further example of a sleep disordered breathing alert system in accordance with embodiments of the invention. In this example, after receiving a signal transmitted from the implantable device 1010, the patient-worn device 1030 transmits a signal to a mobile communications device 1040 such as a cell telephone or pager. The patient-worn device 1030 may also generate and audible alert.

FIG. 10E illustrates yet another embodiment of a sleep disordered breathing alert system in accordance with embodiments of the invention. In this embodiment, the patient-external device 1020 comprises a respiration therapy device, such as a positive airway pressure device. The patient-external device 1020 may comprise, for example, an external breathing therapy device such as a continuous positive airway pressure device (CPAP), bi-level positive airway pressure device (bi-PAP) or other positive airway pressure device, generically referred to herein as xPAP devices.

An xPAP device 1020 develops a positive air pressure that is delivered to the patient's airway through tubing 1052 and mask 1054 connected to the xPAP device 1020. Positive airway pressure devices are often used to treat disordered breathing. The positive airway pressure provided by the xPAP device 1020 acts as a pneumatic splint keeping the patient's airway open and reducing the severity and/or number of occurrences of disordered breathing due to airway obstruction. In addition to delivering breathing therapy, the xPAP device 1020 may provide sensing functionality for sensing respiration through airflow sensors positioned on the mask 1054. In one configuration, the airflow information may be telemetered to the implantable device 1010 for detection of disordered breathing, for example. In another configuration, the implantable device 1010 may determine that the patient is asleep and the patient-external device 1020 may detect sleep disordered breathing.

Following the detection of sleep disordered breathing, the patient-external device 1020 may generate an audible alert through a speaker 1022 or similar device. Other types of alerts, e.g., visual and/or vibratory alerts are also possible. The implantable device 1010 or the patient-external device 1020 may include a memory for storing information about the sleep disordered breathing episodes. The information may be transmitted to a separate computing device 1060 for further analysis.

FIG. 10F illustrates yet another implementation of a disordered breathing alert system in accordance with embodiments of the invention. In this example, the implantable device is configured as a cardiac rhythm management system (CRM) 1090 incorporating circuitry for detecting sleep disordered breathing and a cardiac therapy unit for providing electrical stimulation therapy to the heart. The CRM 1090 and a programmer 1080 are wirelessly coupled for bidirectional communication. The programmer 1080 includes a therapy control unit 1081 that may be used to adjust the therapy provided by the CRM 1090. In various embodiments, the programmer 1080 may be implemented, for example, as a bedside device or a patient-worn or patient-carried device.

Upon detection of sleep disordered breathing, the CRM 1090 transmits a signal to the programmer 1080. The programmer 1080 may be equipped with a speaker 1022 for generating an audible sleep disordered breathing alert and/or a display 1021 for displaying a visual sleep disordered breathing alert in response to the signal. As previously mentioned, the CRM device 1090 may also transmit additional information about the sleep disordered breathing episode to the programmer 1080, e.g., severity, duration, and/or date/time the disordered breathing episodes occurred, for example. The additional information may be stored, displayed, analyzed, transmitted to another device, and/or used for other purposes.

Upon receiving sleep disordered breathing information from the CRM device 1090, the programmer 1080 may communicate with the CRM device 1090 to adjust pacing therapy delivered to the patient. The programmer may communicate with the CRM device to direct the CRM to initiate, modify, or terminate cardiac electrical stimulation therapy. In one implementation, the programmer may communicate with the CRM device to initiate overdrive pacing involving pacing at a rate above a normal sleep rate, for example. In other implementations, the programmer may be used to initiate a particular pacing regimen or to switch from one pacing mode to another pacing mode. In one example, the cardiac pacing regimen may be switched from a dual-chamber pacing mode to a bi-ventricular or other resynchronization mode. In other examples, the pacing mode may be switched to a pacing mode that promotes atrial pacing, or promotes consistent ventricular pacing. In yet another example, the cardiac electrical therapy may involve initiating multi-site electrical stimulation to the heart or changing from one electrical stimulation site to another. The pacing mode may be switched from single chamber to multiple chambers, or the reverse. For example, a bi-ventricular mode may be switched to a left ventricular mode only. Alternatively, a single chamber mode, e.g., LV or RV only, may be switched to a bi-ventricular mode. Other therapy regimens, involving various pacing modes, pacing sites, or non-excitatory electrical stimulations, are possible in connection with providing cardiac electrical therapy for disordered breathing. The type of cardiac electrical therapy beneficial to a patient is highly patient specific and an acceptable or optimal therapy may be determined based on the responses of a particular patient.

A number of the examples presented herein involve block diagrams illustrating functional blocks used to provide a disordered breathing alert system in accordance with embodiments of the present invention. It will be understood by those skilled in the art that there exist many possible configurations in which these functional blocks may be arranged and implemented. The examples depicted herein provide examples of possible functional arrangements used to implement the approaches of the invention.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

1. A sleep disordered breathing alert system, comprising:
a sensing system configured to sense one or more conditions associated with sleep disordered breathing;
an implantable device coupled to the sensing system, the implantable device comprising:
a processor configured to detect disordered breathing occurring during sleep; and
a transmitter configured to transmit a signal if the sleep disordered breathing is detected; and
a patient-external device configured to receive the signal and generate an alert responsive to the detection of the sleep disordered breathing.

2. The system of claim 1, wherein the sensing system comprises a sensor configured to a physiological condition.

3. The system of claim 1, wherein the sensing system comprises a sensor configured to sense a non-physiological condition.

4. The system of claim 1, wherein the implantable device comprises a cardiac device.

5. The system of claim 1, wherein the patient-external device is configured to generate the alert to arouse the patient from sleep.

6. The system of claim 1, wherein:
the patient-external device comprises a sound generating device; and
the alert comprises an audible sound.

7. The system of claim 1, wherein:
the patient-external device comprises a vibration generating device; and
the alert comprises a vibration.

8. The system of claim 1, wherein:
the patient-external device comprises a display device; and
the alert comprises a visual display.

9. The system of claim 1, wherein the patient-external device comprises a mobile communication device.

10. The system of claim 1, wherein the patient-external device comprises a bed-side monitor.

11. The system of claim 1, wherein the patient-external device comprises a therapy device programmer.

12. The system of claim 1, wherein the patient-external device comprises a patient-worn device.

13. The system of claim 1, wherein the patient-worn device comprises an adhesive backed device.

14. The system of claim 1, further comprising a memory configured to store information about the sleep disordered breathing.

15. The system of claim 14, wherein the memory is a component of the patient-external device.

16. The system of claim 14, wherein the memory is a component of the implantable device.

17. The system of claim 1, further comprising a therapy system configured to deliver therapy to treat the sleep disordered breathing.

18. The system of claim 17, wherein the therapy comprises cardiac electrical stimulation therapy.

19. The system of claim 17, wherein the therapy comprises external respiration therapy.

20. The system of claim 17, wherein the therapy system is a component of the implantable device.

21. The system of claim 17, wherein the therapy system is a component of the patient-external device.

22. A method, comprising:
detecting that a patient is asleep;
detecting disordered breathing occurring during sleep; and
generating an alert responsive to the detection of the sleep disordered breathing, wherein at least one of detecting that the patient is asleep and detecting the disordered breathing is performed implantably.

23. The method of claim 22, wherein detecting that the patient is asleep comprises comparing one or more conditions related to sleep to one or more sleep indices.

24. The method of claim 22, wherein detecting the disordered breathing comprises:
sensing respiration patterns of the patient; and
detecting the disordered breathing based on the respiration patterns.

25. The method of claim 22, wherein detecting the disordered breathing comprises:
sensing transthoracic impedance of the patient; and
detecting the disordered breathing based on the transthoracic impedance.

26. The method of claim 22, further comprising transmitting information about the sleep disordered breathing from an implantable device to a patient-external device.

27. The method of claim 22, wherein generating the alert comprises generating the alert using a patient-external device.

28. The method of claim 22, wherein generating the alert comprises generating an audible alert.

29. The method of claim 22, wherein generating the alert comprises generating a vibratory alert.

30. The method of claim 22, wherein generating the alert comprises generating a visual alert.

31. The method of claim 22, further comprising storing information about the disordered breathing.

32. The method of claim 22, further comprising delivering therapy to treat the disordered breathing.

33. A sleep disordered breathing alert system, comprising:
means for detecting that a patient is asleep;
means for detecting disordered breathing occurring during sleep; and
means for generating an alert responsive to the detection of the sleep disordered breathing, wherein at least one of the means for detecting that the patient is asleep and the means for detecting the sleep disordered breathing include an implantable component.

34. The system of claim 33, further comprising means for transmitting information about the disordered breathing from an implantable device to a patient-external device.

35. The method of claim 33, further comprising means for storing information about the disordered breathing.

36. The method of claim 32, further comprising means for delivering therapy to treat the disordered breathing.
